(19) United States
(12) Patent Application Publication
(10) Pub. No.: US 2007/0117987 A1
(43) Pub. Date: May 24, 2007

(54) Process for preparing valsartan

(76) Inventors: Viviana Braude, Kadima (IL); Serguei Fine, Qiriat-Arbaa (IL); Slavik Yurkovsky, Kiryat Gat (IL); Evgeny Tsiperman, Beer-Sheva (IL)

Correspondence Address: KENYON & KENYON LLP, ONE BROADWAY, NEW YORK, NY, 10004, US

(21) Appl. No.: 11/481,736

(22) Filed: Jul. 5, 2006

Related U.S. Application Data
(60) Provisional application No. 60/697,016, filed on Jul. 5, 2005.
(60) Provisional application No. 60/739,214, filed on Nov. 22, 2005.

(51) Int. Cl. C07D 257/02

(52) U.S. Cl. 548/253

(57) ABSTRACT

Provided is a process for preparing valsartan and precursors thereof.

### CROSS-REFERENCE TO RELATED APPLICATIONS

This application claims the benefit of U.S. provisional application Nos. 60/697,016, filed Jul. 5, 2005, and 60/739,214, filed Nov. 22, 2005, herein incorporated by reference.

### FIELD OF THE INVENTION

The present invention relates to a process for preparing Valsartan and precursors thereof.

### BACKGROUND OF THE INVENTION

Valsartan, also known as (S)-N-(1-Carboxy-2-methyl-prop-1-yl)-N-pentanoyl-N-[2′-(1H-tetrazol-5-yl)biphenyl-4-ylmethyl]-amine, has the following structure: 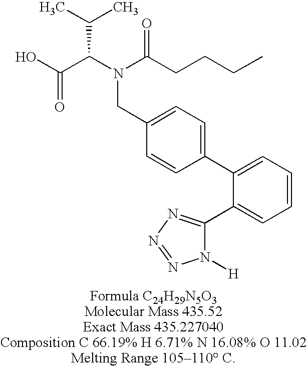
and is marketed as the free acid under the name DIOVAN. DIOVAN is prescribed as oral tablets in dosages of 40 mg, 80 mg, 160 mg and 320 mg of Valsartan.

Valsartan and/or its intermediates are disclosed in various references, including: U.S. Pat. Nos. 5,399,578, 5,965,592, 5,260,325, 6,271,375, WO 02/006253, WO 01/082858, WO 99/67231, WO 97/30036, Peter Bühlmayer, et al., Bioorgan. & Med. Chem. Let., 4(1) 29-34 (1994), Th. Moenius, et al., J. Labelled Cpd. Radiopharm., 43(13) 1245-1252 (2000), and Qingzhong Jia, et al., Zhongguo Yiyao Gongye Zazhi, 32(9) 385-387 (2001).

Valsartan is an orally active specific angiotensin II antagonist acting on the AT1 receptor subtype. Valsartan is prescribed for the treatment of hypertension. U.S. Pat. No. 6,395,728 is directed to use of Valsartan for treatment of diabetes related hypertension. U.S. Pat. Nos. 6,465,502 and 6,485,745 are directed to treatment of lung cancer with Valsartan. U.S. Pat. No. 6,294,197 is directed to solid oral dosage forms of Valsartan.

Valsartan methylester can be prepared by solid state synthesis, as disclosed by J. D. Revell and A. Ganesan, J. Chem. Soc., Chem. Commun., 2004, (17), 1916-1917, depicted in the following scheme: 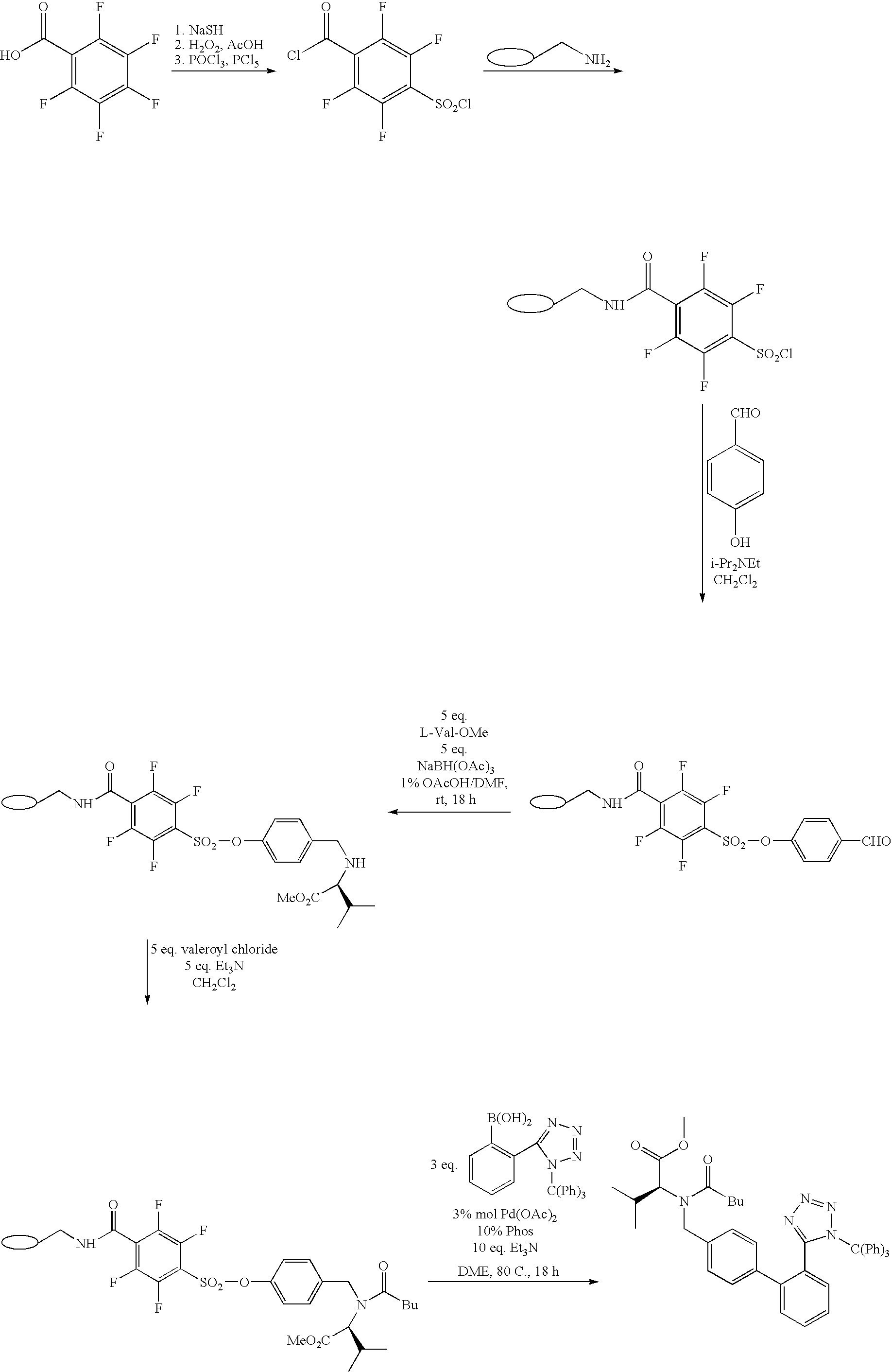
The key step of the above synthesis is the preparation of the biphenyl moiety, which is done in the last step while removing the solid-phase linker. The solid-phase and the linker that need also to be prepared, affect the cost of such process, thus the process is not economic, not efficient and cannot be adapted to an industrial scale.

Therefore, there is a need in the art for an improved synthetic process for the preparation of Valsartan and precursors of Valsartan.

### SUMMARY OF THE INVENTION

In one embodiment, the present invention provides a novel compound of formula I: 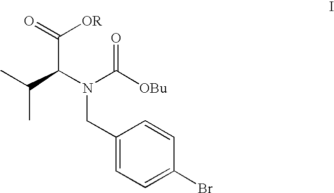
which can be used as an intermediate for preparing Valsartan.

Preferably, R is an optionally substituted C₁ to C₇ straight, or branched alkyl group, or an optionally substituted C₆ to C₇ aromatic group (such as a phenyl).

In another embodiment, the present invention provides a process for preparing formula I comprising the steps of:
(a) combining a C₁-C₇ straight, branched or aromatic, optionally substituted, alkyl or phenyl esters of valine and the compound of formula Ib 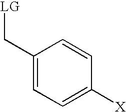
   with an aprotic organic solvent in the presence of a first base to obtain a mixture;
(b) maintaining the obtained reaction mixture at a temperature of about ambient to about 160° C.;
(c) recovering the intermediate of formula Ia; 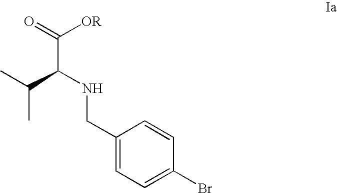
(d) combining the recovered intermediate of formula Ia with an aprotic organic solvent, a second base and valeroyl halide to obtain a mixture;
(e) maintaining the mixture of step (d) at a temperature of about 0° C. to about 70° C.; and
(f) recovering of the compound of formula I.

In another embodiment, the present invention provides a process for preparing the compound of formula III 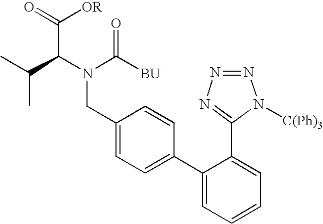
comprising the steps of:
(a) combining a solution of a metal catalyst or appropriate components for preparing a metal catalyst in situ in an organic solvent, with 2-(1-trityl-1H-tetrazol-5-yl)phenylboronic acid of formula II, 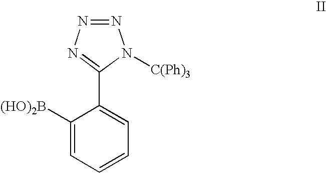
   water, and a base with the compound of formula I, to obtain a mixture;
(b) maintaining the obtained mixture at a temperature of about 40° C. to about 150° C. for about 1 hour to about 12 hours; and
(c) recovering the compound of formula III.

### BRIEF DESCRIPTION OF THE DRAWINGS

FIG. 1 illustrates the synthesis of (S)-methyl-2-(4-bromobenzylamino)-3-methylbutanoate.

FIG. 2 illustrates the synthesis of (S)-methyl-2-(N-(4-bromobenzyl)pentanamido)-3-methylbutanoate.

FIG. 3 illustrates the synthesis of (S)-3-methyl-2-{pentanoyl-[2′-(1-trityl-1H-tetrazol-5-yl)-biphenyl-4-yl methyl]amino}butyric acid, methyl ester.

FIG. 4 illustrates the synthesis of trityl valsartan (“TVLS”).

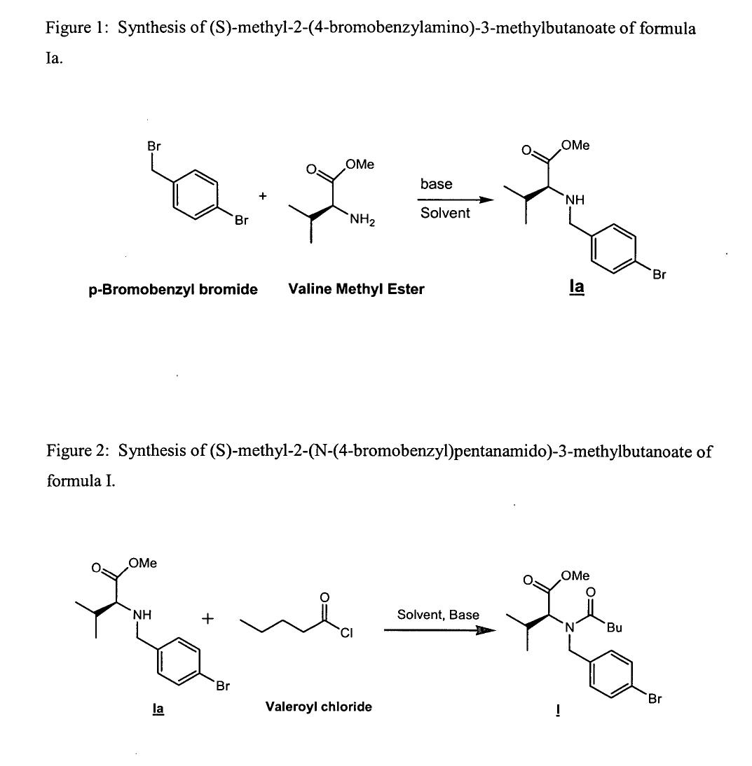

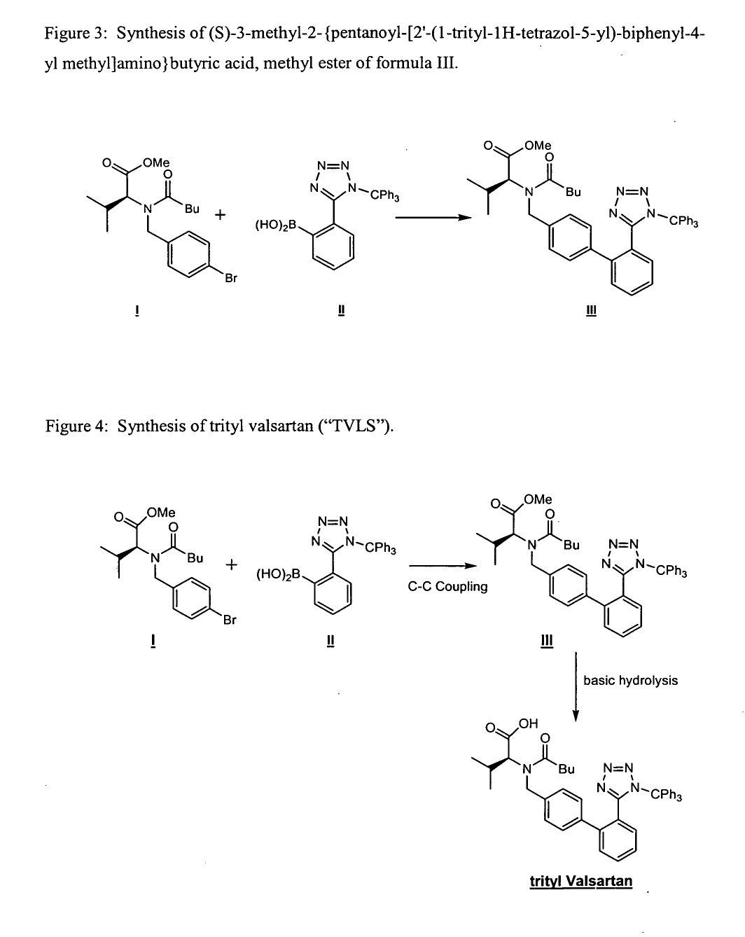

### DETAILED DESCRIPTION OF THE INVENTION

The present invention provides a novel compound of formula I: 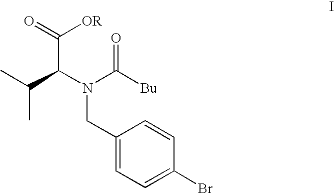
which can be used as an intermediate for preparing Valsartan.

Preferably, R is selected from the group consisting of: C₁ to C₇ straight, branched or aromatic, optionally substituted, alkyl or phenyl.

More preferably, R is selected from the group consisting of: C₁ to C₄ straight or branched alkyl, most preferably R is methyl or t-butyl.

Wherein R is methyl the compound is (S)-methyl-2-(N-(4-bromobenzyl)pentanamido)-3-methylbutanoate, having the formula:

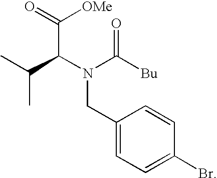

The present invention also provides a process for preparing formula I comprising preparing the intermediate of formula Ia; 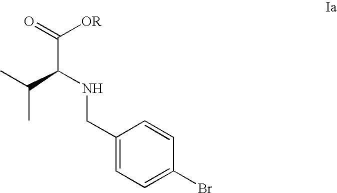
and converting the intermediate of formula Ia to formula I.

Preferably, R is selected from the group consisting of: C₁ to C₇ straight, branched or aromatic, optionally substituted, alkyl or phenyl.

More preferably, R is selected from the group consisting of: C₁ to C₄ straight or branched alkyl, most preferably R is methyl or t-butyl.

Wherein R is methyl the compound is (S)-methyl-2-(N-(4-bromobenzylamido)-3-methylbutanoate, having the formula:

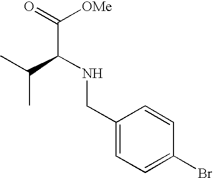

In the process of the invention, the intermediate of formula Ia is prepared by combining a C₁-C₇ straight, branched or aromatic, optionally substituted, alkyl or phenyl esters of valine and the compound of the formula Ib:

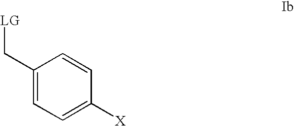

Wherein LG and X are leaving groups selected from the group consisting of: halides and sulfonyloxy groups.

with an aprotic organic solvent in the presence of a first base to obtain a mixture.

Preferably, the valine ester used in step (a) is selected from the group consisting of: C₁ to C₄ straight or branched alkyl. More preferably, the valine ester is either methyl ester or t-butyl ester.

Preferably, the valine ester used in step (a) is in a form of a free base that is obtained from the commercially available hydrochloride salt.

Preferably, the sulfonyloxy leaving groups of formula Ib are selected from the group consisting of: methylsulfonyloxy, p-nitrobenzenesulfonyloxy, benzenesulfonyloxy, p-toluenesolfonyloxy, trifluoromethanesulfonate, nonafluorobutanesulfonate and 2,2,2-trifluoroethanesulfonate.

More preferably, a p-halobenzyl halide is used. Most preferably, the p-halobenzyl halide is p-bromobenzyl bromide, which is commercially available.

Preferably, the first base is an inorganic or an organic base. The inorganic base may be an inorganic salt derived from a reaction between an alkaline base or an alkaline earth metal base with a weak acid. Preferably the inorganic salt is a carbonate or phosphate of an alkali metal or alkaline earth metal, such as potassium carbonate or Na₃PO₄. The organic base may be one having a weak nucleophilic character. Preferred organic bases include tertiary amines, especially tri(C₁ to C₆ alkyl)amines, wherein the alkyl group may be the same or different. Particularly preferred are tri(C₁ to C₃ alkyl)amines, such as triethylamine.

Preferably, the amount of the first base is of about 1.5 to about 40 mole, more preferably, about 3 to about 8 mole per mole of p-halobenzyl halide.

Preferably, the aprotic organic solvent is selected from the group consisting of nitriles, amides, ethers, esters, ketones, aliphatic halogenated hydrocarbons and C₆₋₈ aromatic hydrocarbons. A preferred nitrile is acetonitrile. A preferred amide is N,N-dimethylformamide. A preferred ether is tetrahydrofuran. A preferred ester is ethyl acetate. A preferred ketone is acetone. A preferred aliphatic halogenated hydrocarbon is dichloromethane. A preferred C₆₋₈ aromatic hydrocarbon is toluene.

Preferably, the obtained mixture is maintained at a temperature of about ambient to about 160° C. until one of the reagents has disappeared.

The intermediate of formula Ia is then recovered from the reaction mixture.

The intermediate of formula Ia may be recovered by cooling the mixture to a temperature below 30° C., preferably to a temperature of about 0° C., filtering off the obtained precipitate, washing the precipitate with an organic solvent and evaporation of the filtrates.

The intermediate of formula Ia is obtained by the above process in a purity of about 90% to about 100% area by HPLC, preferably, of about 97% to about 100% area by HPLC.

The intermediate of formula Ia is then converted to the compound of Formula I by the following process.

The intermediate of formula Ia is combined with an aprotic organic solvent, a second base and valeroyl halide to obtain a mixture.

Preferably, the second base is either an inorganic base such as alkali or alkaline earth metal base or an organic base. Preferably, the inorganic base is potassium carbonate. Preferably, the organic base is a tertiary amine, more preferably, triethylamine. Optionally, the first base and the second base can be the same, depending on the solvent.

Preferably, the amount of the second base is of about 1.5 to about 40 mole, more preferably, about 2 to about 4 mole per mole of the intermediate of formula Ia.

Preferably, the valeroyl halide is valeroyl chloride, which is commercially available.

Preferably, formula Ia, an aprotic solvent and a second base are combined to obtain a mixture, prior to the addition of the valeroyl halide, for safety and purity reasons. When the valeroyl halide is added to the above mixture, it is added in a drop-wise manner.

The obtained mixture is then maintained at a temperature of about 0° C. to about 70° C. until the starting material disappears.

The obtained formula I is then recovered from the reaction mixture by any method known in the art, such as filtration, washing the filter cake with another portion of the same solvent, and evaporation of the combined filtrates The intermediate of formula I is obtained by the above process in a purity of about 90% to about 100% area by HPLC, preferably, of about 97% to about 100% area by HPLC.

In a preferred embodiment, the present invention provides a process for preparing formula I comprising the steps of:

(a) combining a C₁-C₇ straight, branched or aromatic, optionally substituted, alkyl or phenyl esters of valine and the compound of formula Ib 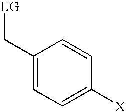
with an aprotic organic solvent in the presence of a first base to obtain a mixture;
(b) maintaining the obtained reaction mixture at a temperature of about ambient to about 160° C.;
(c) recovering the intermediate of formula Ia; 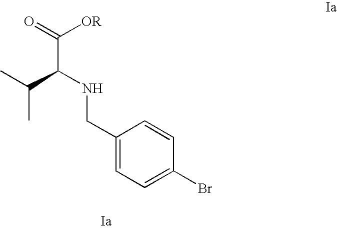
(d) combining the recovered intermediate of formula Ia with an aprotic organic solvent, a second base and valeroyl halide to obtain a mixture;
(e) maintaining the mixture of step (d) at a temperature of about 0° C. to about 70° C.; and
(f) recovering the compound of formula I.

Preferably, the valine esters, the leaving groups, the solvents and the first and second bases, are as described above.

Also provided by the present invention, is a process for preparing Valsartan comprising the steps of preparing formula I and further converting to Valsartan.

The compound of formula III, 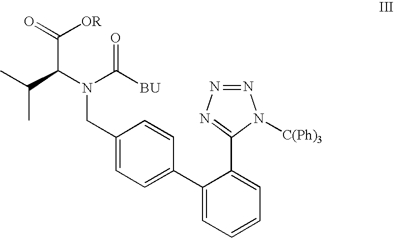
wherein R is defined as for compound Ia and I above, is typically prepared by a C—C coupling reaction. Such a synthetic step is known as a Suzuki coupling reaction and is disclosed in N. Miyaura et al., Tetrahedron Letters, 1979, 3437 and in N. Miyaura and A. Suzuki, J. Chem. Soc., Chem. Commun., 1979, (19), 866-867. The Suzuki coupling can be carried out in a homogeneous mixture or two-phase system having first and second liquid phases. Moreover, it is known to a person skilled in the art that each substrate requires different and appropriate reaction conditions. Accordingly, the Suzuki reaction disclosed by J. D. Revell and A. Ganesan, J. Chem. Soc., Chem. Commun., 2004, (17), 1916-1917 suffers from a disadvantage that only a specific catalyst, which is usually expensive, can be applied under the solid phase conditions.

The Suzuki coupling reaction of the present invention is conducted under mild conditions. Furthermore, it applies a single solvent system unlike the prior art processes, which apply a mixture of solvents. Also, the catalyst is prepared in-situ, the base is easy to handle and is not hazardous and does not lead to side reactions. Thus, the Suzuki-coupling reaction conducted under the conditions of the present invention is cheaper, not hazardous and suitable for larger scales.

The present invention further provides a process for preparing the compound of formula III comprising the steps of:
(a) combining a solution of a metal catalyst or appropriate components for preparing a metal catalyst in situ in an organic solvent, with 2-(1-trityl-1H-tetrazol-5-yl) phenylboronic acid of formula II, 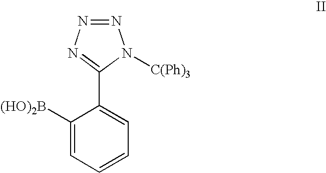
   water, and a base with the compound of formula I, to obtain a mixture;
(b) maintaining the obtained mixture at a temperature of about 40° C. to about 150° C. for about 1 hour to about 12 hours; and
(c) recovering the compound of formula III.

Wherein R is methyl, the recovered compound is (S)-3-methyl-2-{pentanoyl-[2′-(1-trityl-1H-tetrazol-5-yl)-biphenyl-4-yl methyl]amino}butyric acid, methyl ester, having the formula:

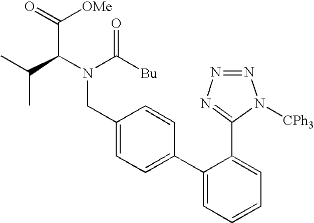

2-(1-trityl-1H-tetrazol-5-yl)phenylboronic acid of formula II may be prepared for example, according to the process disclosed in US 2004/0192713.

Preferably, components of a metal catalyst are used in step (a), so the catalyst is formed in situ.

Preferably, the metal containing component of the catalyst is either Pd(II)(OAc)₂ or Pd(II)Cl₂, more preferably, Pd(II)(OAc)₂.

Preferably, the amount of the metal catalyst used in step (a) is of about 0.005 to about 0.1 mole per mole of formula I, more preferably, of about 0.01 to about 0.02 mole per mole of formula I.

Preferably, when a metal containing component is used in step (a) for preparing the catalyst in situ, it is combined with a trivalent phosphorous derivative prior to the addition of 2-(1-trityl-1H-tetrazol-5-yl)phenylboronic acid of formula II.

Preferably, the trivalent phosphorous derivative is triphenyl phosphine.

Preferably, the organic solvent is a single solvent. The single organic solvent is either an aromatic hydrocarbon or heterocyclic aromatic hydrocarbon. A preferred aromatic hydrocarbon is toluene, xylene or tetraline. A preferred heterocyclic aromatic hydrocarbon is pyridine. Toluene is particularly preferred as a single solvent when the metal catalyst is a palladium complex.

Optionally, the organic solvent is in a mixture with ethers such as dimethoxyethane (DME) and tetrahydrofuran (THF).

Preferably, the amount of water used in step (a) is of about 2.5 mole per mole of formula I.

Preferably, the base is an inorganic base, more preferably, an alkali or alkaline earth metal hydroxide, a carbonate or a phosphate. Most preferably, the base is potassium carbonate.

Preferably, the amount of the base used in step (a) is of about 1 to about 20 mole, more preferably, about 2 to about 4 mole per mole of formula I.

Preferably, the temperature of step (b) is of about 70° C. to about 120° C.

The present invention provides a process for preparing valsartan comprising the steps of preparing the compound of formula III and further converting to valsartan.

The process of the present invention provides valsartan by initially preparing the biphenyl moiety which serves throughout the synthesis as a building block. Moreover, it uses an alkyl, phenyl or benzyl ester protecting group, thus allowing for a smooth removal under relative mild conditions. Thus, fewer steps are conducted, the reagents used are not hazardous and the cost is reduced, making the process suitable for larger scale.

The present invention also provides a process for the preparation of Valsartan comprising the steps of;
(a) combining a C₁-C₇ straight, branched or aromatic, optionally substituted, alkyl or phenyl esters of valine and the compound of formula Ib 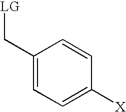
   with an aprotic organic solvent in the presence of a first base to obtain a mixture;
(b) maintaining the obtained reaction mixture at a temperature of about ambient to about 160° C., (until one of the reagents has disappeared);
(c) recovering the intermediate of formula Ia; 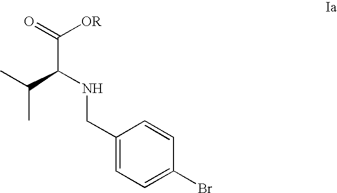
(d) combining the recovered intermediate of formula Ia with an aprotic organic solvent, a second base and valeroyl halide to obtain a mixture;
(e) maintaining the mixture of step (d) at a temperature of about 0° C. to about 70° C., (until the starting material disappears);
(f) recovering the compound of formula I;
(g) combining a solution of a metal catalyst or appropriate components for preparing a metal catalyst in situ in an organic solvent with 2-(1-trityl-1H-tetrazol-5-yl)phenylboronic acid of formula II, water, a base and the compound of formula I, to obtain a mixture, and
(h) maintaining the obtained mixture at a temperature of about 40° C. to about 150° C. for about 1 hour to about 12 hours;
(i) recovering the compound of formula III;
(j) combining the compound of formula III with a base to obtain (S)-3-methyl-2-{pentanoyl-[2′-(1-trityl-1H-tetrazol-5-yl)-biphenyl-4-yl methyl]amino}butyric acid (trityl Valsartan) of formula IV; and 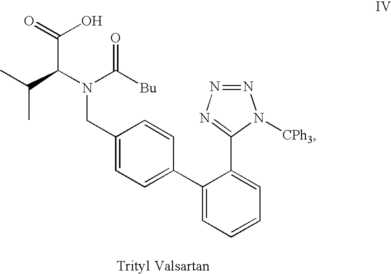
(k) combining trityl Valsartan of formula IV with an acid to obtain Valsartan.

Optionally, Valsartan can be directly obtained from the compound of formula III by acidic or basic hydrolysis.

Having described the invention with reference to certain preferred embodiments, other embodiments will become apparent to one skilled in the art from consideration of the specification. The invention is further defined by reference to the following examples. The examples are set forth in aid in the understanding of the invention, but are not intended to, and should not be construed to, limit its scope in any way. It will be apparent to those skilled in the art that many modifications, both to materials and methods, may be practiced without departing from the scope of the invention. The examples do not include detailed descriptions of conventional methods. Such methods are well known to those or ordinary skill in the art and are described in numerous publications. All references mentioned herein are incorporated by reference in their entirety.

### EXAMPLES

### Example 1

### Preparation of Ia

Valine methyl ester (as a free base) (25 g, 0.191 mole, 1.2 eq.) and potassium carbonate (175.9 g, 1.27 mole, 8 eq.) were mixed with 400 ml of acetonitrile. Then a solution of p-bromobenzyl bromide (39.75 g, 0.159 mole) in 200 ml acetonitrile was added. The reaction mixture was heated to 70° C. After 2 hours, the reaction was completed. The mixture was cooled to 0° C. The precipitate was filtered and washed with fresh acetonitrile. 50.3 g of the compound la were isolated and used as is in the next step. The yield was 99%, with a purity of 98.5% area by HPLC.

¹H NMR (CDCl₃): δ 0.89 (t 6H), 1.81 (s, H), 1.87 (m, 1H), 2.93 (d, 1H), 3.47 (d, 1H), 3.74 (d,1H), 7.17 (d, 2H), 7.36 (d, 2H).

### Example 2

### Preparation of (S)-methyl-2-(N-(4-bromobenzyl)pentanamido)-3-methylbutanoate of formula I

A three necked flask was charged under nitrogen, with compound Ia (5 g, 0.016 mole), K₂CO₃ (6.9 g, 0.05 mole, 3 eq.) and 60 ml of acetonitrile. Valeroyl chloride (5.02 g, 0.042 mole, 2.5 eq.) was added drop-wise for 7 minutes while the temperature was maintained below 35° C. The reaction was completed after 2 hours. The precipitate was filtered and dried with sodium sulfate. 8.8 g compound I were obtained having a purity of 97.4% area by HPLC.

### Example 3

### Preparation of (S)-3-methyl-2-{pentanoyl-[2′-(1-trityl-1H-tetrazol-5-yl)-biphenyl-4-yl methyl]amino}butyric acid, methyl ester of formula III

A mixture of 40 ml of DME and 10 ml of THF was degassed by vacuum/nitrogen purges followed by the addition of Ph₃P (0.14 g, 0.00052 mole, 0.08 eq.). After the Ph₃P was dissolved, Pd(OAc)₂ (3.3 mg, 0.00013 mole, 0.02 eq.) was added and the mixture was further degassed (2 times) and stirred for 30 min at room temperature. Then, II, 2-(1-trityl-1H-tetrazol-5-yl)phenylboronic acid (3.51 g, 0.0068 mole, 1.05 eq.) was suspended in the reaction mixture and the stirring was continued. After stirring for 10 min at room temperature, water (0.3 g, 0.0163 mole, 2.5 eq.) was added, and the slurry was stirred for an additional 30 min. Powdered K₂CO₃ (2.25 g, 0.0163 mole, 2.5 eq,) and I (2.5 g, 0.0065 mole) were then added sequentially and the resulting mixture was degassed (3 times), and refluxed (approx. 80° C.) for 3 hours. The reaction was monitored by TLC (Hex/EtOAc 2:1). The solvents were evaporated under reduced pressure. Toluene (20 mL) and water (20 mL) were then added. The organic and aqueous phases were separated. After separation, the aqueous phase was extracted with toluene (50 mL) and the combined organic phases were washed with water and brine, dried over Na₂SO₄, and evaporated under reduced pressure to give 3.6 g of III as an oil having a purity of 83% area by HPLC.

### Example 4

### Preparation of (S)-3-methyl-2-{pentanoyl-[2′-(1-trityl-1H-tetrazol-5-yl)-biphenyl-4-yl methyl]amino}butyric acid, methyl ester of formula III in the presence of Pd(OAc)₂ and PPh₃

Toluene (240 ml) was degassed by purging nitrogen for 0.5 hour followed by the addition of Ph₃P (0.41 g, 0.00156 mole, 0.08 eq.). After the Ph₃P was dissolved, Pd(OAc)₂ (70 mg, 0.00031 mole, 0.01 eq.) was added and the mixture was further degassed for another 0.5 hours. Then II, 2-(1-trityl-1H-tetrazol-5-yl)phenylboronic acid (16.8 g, 0.033 mole, 1.06 eq.) was introduced into the reaction mixture and the stirring was continued. After stirring for 10 min at room temperature, water (1.4 g, 0.078 mole, 2.5 eq.) was added and the slurry was stirred for additional 30 min. Powdered K₂CO₃ (10.8 g, 0.078 mole, 2.5 eq.) and 1 (12 g, 0.031 mole) were then added sequentially and the mixture was further degassed for a half an hour. The mixture was then heated, under nitrogen atmosphere, to reflux and maintained at reflux until I disappeared (1.5-2 hours). When the reaction was completed, the mixture was cooled to ambient temperature and 240 ml of water were added. The organic and aqueous phases were separated. After separation, the aqueous phase was washed with 240 ml of toluene and the combined organic phases were washed with water and brine, dried over Na₂SO₄ and evaporated under reduced pressure. 26.87 g of yellow oily product III having a purity of 83% area by HPLC, were obtained.

### Example 5

### Preparation of (S)-3-methyl-2-{pentanoyl-[2′-(1-trityl-1H-tetrazol-5-yl)-biphenyl-4-yl methyl[amino}butyric acid methyl ester of formula III in the presence of commercially available Pd(PPh₃)₄

Toluene (20 ml) was degassed by purging nitrogen for 0.5 hour followed by a subsequent addition of Pd(PPh₃)₄ (150 mg, 0.00013 mole, 0.02 eq.) and 2-(1-trityl-1H-tetrazol-5-yl)phenylboronic acid (II) (3.5 g, 0.0068 mole, 1.05 eq.). The mixture was further degassed for 0.25 hours and then water (0.3 g, 0.0163 mole, 2.5 eq.) were added and the slurry was stirred for additional 20 min. Powdered K₂CO₃ (2.25 g, 0.0163 mole, 2.5 eq.) and 1 (2.5 g, 0.0065 mole) were added to the reaction mixture followed by further degassing for 0.25 hour. Subsequently, the mixture was heated, under nitrogen atmosphere, to reflux and maintained at reflux until I disappeared (1.5-2 hours). When the reaction was complete, the mixture was cooled to ambient temperature and washed with 50 ml of water. The organic and aqueous phases were separated. After separation, the aqueous phase was washed with 25 ml of toluene and the combined organic phases were washed with water and brine, dried over Na₂SO₄ and evaporated under reduced pressure. 5.5 g of yellow oily product III having a purity of 72.5% area by HPLC were obtained.

Chromatographic Purity Determination by Reversed Phase HPLC

The chromatographic purity determination of TVLS Preparation is based on a reversed phase HPLC method using a C8 5-um silica stationary phase and a gradient mobile phase containing Acetonitrile and 20 mM K2HPO4 Buffer pH 7.8.

| | |
|---|---|
| Column: | Zorbax Eclipse XDB C8 5 μm 80 Å 250 mm × 4.6 mm |
| | (Agilent) |
| Mobile Phase: | A: 20 mM di-Potassium Hydrogen Phosphate Buffer |
| | solution of pH 7.8 |
| | prepared in 1-L volumetric flask by dissolving 3.48 g |
| | K₂HPO₄ in HPLC grade water, followed |
| | by adjustment of pH to 7.8 with conc. H₃PO₄ |
| | and making up to volume with HPLC grade water. |
| | B: Acetonitrile (gradient grade) |
| Gradient: | 0 min - 85% A/15% B, 3 min - 50% A/50% B |
| | 15 min - 30% A/70% B, 40 min - 30% A/70% B |
| | 45 min - 25% A/75% B, 60 min - 25% A/75% A |
| Flow rate: | 1.0 mL/min |
| Detector: | PDA/UV 1t 220 nm |
| Column Temp.: | 25° C. |
| Sample Temp.: | 10° C. |
| Injection Vol.: | 10 μL |
| Diluent: | 100% Acetonitrile |

What is claimed is:

1. A compound of formula I: 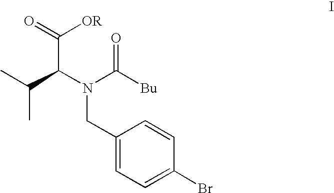
wherein R is an optionally substituted C₁ to C₇ straight, or branched alkyl group, or an optionally substituted C₆ to C₇ aromatic group.

2. The compound of claim 1, wherein R is selected from the group consisting of:
C₁ to C₄ straight or branched alkyl.

3. The compound of claim 2, wherein R is methyl or t-butyl.

4. The compound of claim 3, wherein R is methyl, having the formula:

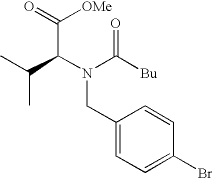

5. A process for preparing the compound of claim 1 comprising:
a. providing a compound of formula Ia; 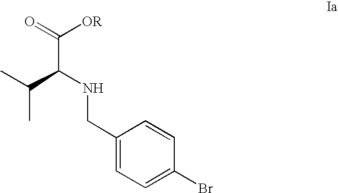
;and
b. converting the compound of formula Ia to formula I.

6. The process of claim 5, wherein in step a), R is an optionally substituted C₁ to C₇ straight, or branched alkyl group, or an optionally substituted C₆ to C₇ aromatic group.

7. The process of claim 6, wherein R is selected from the group consisting of: C₁ to C₄ straight or branched alkyl.

8. The process of claim 7, wherein R is methyl or t-butyl.

9. A process for preparing the compound of claim 1 comprising:
(a) combining an optionally substituted C₁ to C₇ straight, or branched alkyl ester, or an optionally substituted C₆ to C₇ aromatic ester of valine and the compound of formula Ib 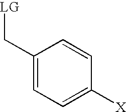
wherein LG and X are leaving groups selected from the group consisting of:
halides and sulfonyloxy groups
with an aprotic organic solvent in the presence of a first base to obtain a mixture;
(b) maintaining the obtained reaction mixture at a temperature of about ambient to about 160° C.;
(c) recovering the intermediate of formula Ia; 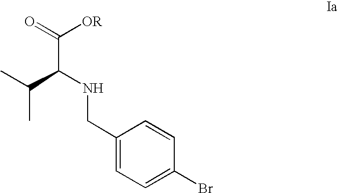
(d) combining the recovered intermediate of formula Ia with an aprotic organic solvent, a second base and valeroyl halide to obtain a mixture;
(e) maintaining the mixture of step (d) at a temperature of about 0° C. to about 70° C.; and
(f) recovering the compound of formula I.

10. The process of claim 9, wherein in step a) the valine ester (a) is selected from the group consisting of: C₁ to C₄ straight or branched alkyl.

11. The process of claim 10, wherein the valine ester is either methyl ester or t-butyl ester.

12. The process of claim 9, wherein the valine ester used in step (a) is in a form of a free base obtained from hydrochloride salt.

13. The process of claim 9, wherein the sulfonyloxy leaving groups in step a) are selected from the group consisting of: methylsulfonyloxy, p-nitrobenzenesulfonyloxy, benzenesulfonyloxy, p-toluenesolfonyloxy, trifluoromethanesulfonate, nonafluorobutanesulfonate and 2,2,2-trifluoroethanesulfonate.

14. The process of claim 9, wherein in step a), a p-halobenzyl halide is used.

15. The process of claim 14, wherein the p-halobenzyl halide is p-bromobenzyl bromide.

16. The process of claim 9, wherein in step a) the first base is an inorganic or an organic base.

17. The process of claim 16, wherein the inorganic base is an inorganic salt derived from a reaction between an alkaline base or an alkaline earth metal base with a weak acid.

18. The process of claim 17, wherein the inorganic salt is a carbonate or phosphate of an alkali metal or alkaline earth metal.

19. The process of claim 18, wherein the inorganic salt is potassium carbonate or Na₃PO₄.

20. The process of claim 16, wherein the organic base is one having a weak nucleophilic character.

21. The process of claim 16, wherein the organic base is tertiary amine.

22. The process of claim 21, wherein the tertiary amine is tri(C₁ to C₆ alkyl)amine wherein the alkyl group may be the same or different.

23. The process of claim 22, wherein the tertiary amine is tri(C₁ to C₃ alkyl)amine.

24. The process of claim 23, wherein the tertiary amine is triethylamine.

25. The process of claim 14, wherein the amount of the first base is of about 1.5 to about 40 mole per mole of p-halobenzyl halide.

26. The process of claim 25, wherein wherein the amount of the first base is of about 3 to about 8 mole per mole of p-halobenzyl halide.

27. The process of claim 9, wherein the aprotic organic solvent in step a) or d) is selected from the group consisting of nitrites, amides, ethers, esters, ketones, aliphatic halogenated hydrocarbons and C₆₋₈ aromatic hydrocarbons.

28. The process of claim 27, wherein the aprotic organic solvent is selected from the group consisting of: acetonitrile, N,N-dimethylformamide, tetrahydrofuran, ethyl acetate, acetone, dichloromethane and toluene.

29. The process of claim 9, wherein in step b) the obtained mixture is maintained at until one of the reagents has disappeared.

30. The process of claim 9, wherein in step c) the intermediate of formula Ia is obtained in a purity of about 90% to about 100% area by HPLC.

31. The process of claim 30, wherein the intermediate of formula Ia is obtained in a purity of about 97% to about 100% area by HPLC.

32. The process of claim 9, wherein the second base in step b) is either an inorganic base such as alkali or alkaline earth metal base or an organic base.

33. The process of claim 32, wherein the inorganic base is potassium carbonate.

34. The process of claim 32, wherein the organic base is a tertiary amine.

35. The process of claim 34, wherein the organic base is triethylamine.

36. The process of claim 9, wherein the amount of the second base is of about 1.5 to about 40 mole per mole of the intermediate of formula Ia.

37. The process of claim 36, wherein the amount of the second base is of about 2 to about 4 mole per mole of the intermediate of formula Ia.

38. The process of claim 9, wherein the valeroyl halide in step d) is valeroyl chloride.

39. The process of claim 9, wherein in step d), formula Ia, an aprotic solvent and a second base are combined to obtain a mixture, prior to the addition of the valeroyl halide.

40. The process of claim 9, wherein in step e) the mixture is maintained until the starting material disappears.

41. The process of claim 9, wherein the compound of formula I is obtained in a purity of about 90% to about 100% area by HPLC.

42. The process of claim 41, wherein the compound of formula I is obtained in a purity of about 97% to about 100% area by HPLC.

43. A process for preparing Valsartan comprising the steps of:
a) providing the compound of formula I; and
b) converting the compound of formula I to Valsartan.

44. A process for preparing the compound of formula III: 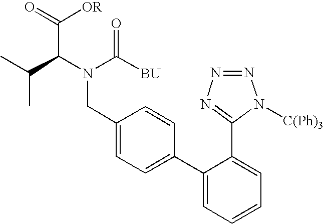
wherein R is as defined in claims 1-3,
comprising the steps of:
a) combining a solution of a metal catalyst or appropriate components for preparing a metal catalyst in situ in an organic solvent, with 2-(1-trityl-1H-tetrazol-5-yl)phenylboronic acid of formula II, 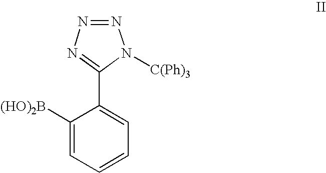
water, and a base with the compound of formula I, to obtain a mixture;
b) maintaining the obtained mixture at a temperature of about 40° C. to about 150° C. for about 1 hour to about 12 hours; and
c) recovering the compound of formula III.

45. The process of claim 44, wherein components of a metal catalyst are used in step (a).

46. The process of claim 45, wherein the metal containing component of the catalyst is either Pd(II)(OAc)₂ or Pd(II)Cl₂.

47. The process of claim 46, wherein the metal containing component of the catalyst is Pd(II)(OAc)₂.

48. The process of claim 44, wherein the metal catalyst used in step (a) is of about 0.005 to about 0.1 mole per mole of formula I.

49. The process of claim 48, wherein the metal catalyst used is of about 0.01 to about 0.02 mole per mole of formula I.

50. The process of claim 45, wherein the metal containing component is combined with a trivalent phosphorous derivative prior to the addition of 2-(1-trityl-1H-tetrazol-5-yl)phenylboronic acid.

51. The process of claim 50, wherein the trivalent phosphorous derivative is triphenyl phosphine.

52. The process of claim 44, wherein the organic solvent is a single solvent.

53. The process of claim 52, wherein the single organic solvent is either an aromatic hydrocarbon or heterocyclic aromatic hydrocarbon.

54. The process of claim 53, wherein the single organic solvent is selected from the group consisting of: toluene, xylene, tetraline and pyridine.

55. The process of claim 54, wherein the single organic solvent is toluene.

56. The process of claim 44, wherein the amount of water used in step (a) is of about 2.5 mole per mole of formula I.

57. The process of claim 44, wherein the base is an inorganic base.

58. The process of claim 57, wherein the inorganic base is an alkali or alkaline earth metal hydroxide, a carbonate or a phosphate.

59. The process of claim 58, wherein the inorganic base is potassium carbonate.

60. The process of claim 44, wherein the amount of the base used in step (a) is of about 1 to about 20 mole per mole of formula I.

61. The process of claim 60, wherein the amount of the base used is of about 2 to about 4 mole per mole of formula I.

62. The process of claim 44, wherein the temperature of step (b) is of about 70° C. to about 120° C.

63. A process for preparing Valsartan comprising the steps of:
a) providing the compound of formula III; and
b) converting the compound of formula III to Valsartan.

64. A process for preparing the compound of formula III comprising the steps of
a) combining an optionally substituted C₁ to C₇ straight, or branched alkyl group, or an optionally substituted C₆ to C₇ aromatic group, and the compound of formula Ib 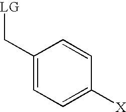
with an aprotic organic solvent in the presence of a first base to obtain a mixture;
b) maintaining the obtained reaction mixture at a temperature of about ambient to about 160° C., (until one of the reagents has disappeared);
c) recovering the intermediate of formula Ia; 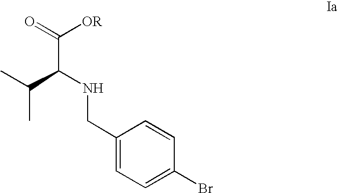
d) combining the recovered intermediate of formula Ia with an aprotic organic solvent, a second base and valeroyl halide to obtain a mixture;
e) maintaining the mixture of step (d) at a temperature of about 0° C. to about 70° C., (until the starting material disappears);
f) recovering the compound of formula I;
g) combining a solution of a metal catalyst or appropriate components for preparing a metal catalyst in situ in an organic solvent with 2-(1-trityl-1H-tetrazol-5-yl)phenylboronic acid of formula II, water, a base and the compound of formula I, to obtain a mixture, and
h) maintaining the obtained mixture at a temperature of about 40° C. to about 150° C. for about 1 hour to about 12 hours;
i) recovering the compound of formula III.

65. The process of claim 64, further comprising:
a) combining the compound of formula III with a base to obtain (S)-3-methyl-2-{pentanoyl-[2′-(1-trityl-1H-tetrazol-5-yl)-biphenyl-4-yl methyl]amino}butyric acid (trityl Valsartan) of formula IV; and 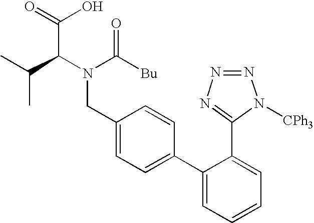
b) combining trityl Valsartan of formula IV with an acid to obtain Valsartan.

66. The process of claim 64, further comprising subjecting the compound of formula III to hydrolysis under acidic conditions, to obtain Valsartan.

67. The process of claim 64, further comprising subjecting the compound of formula III to hydrolysis under basic conditions, to obtain Valsartan.
